(19) United States
(12) Patent Application Publication
(10) Pub. No.: US 2005/0202017 A1
(43) Pub. Date: Sep. 15, 2005

(54) Hb-954 as a target for modulating angiogenesis

(76) Inventors: Katharine H Dixon, Olney, MD (US); Gene Liau, Wayland, MA (US); Cynthia Serdikoff, Collegeville, PA (US)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/525,456

(22) PCT Filed: Aug. 29, 2003
(86) PCT No.: PCT/EP03/09619

§ 371 Date: May 10, 2005

Related U.S. Application Data
(60) Provisional application No. 60/185,942, filed on Feb. 29, 2000.

(51) Int. Cl. G01N 33/574; A61K 39/395; A61K 48/00; C07K 16/30

(52) U.S. Cl. 424/155.1; 435/7.23; 530/388.8; 514/44

(57) ABSTRACT

The invention describes assays for the identification of compounds useful for the modulation of angiogenesis. The methods of the invention involve cell-free and cell-based assays that identify compounds which bind to and/or activate or inhibit the activity of HB-954, a G protein-coupled receptor, optionally followed by an in vivo assay of the effect of the compound on angiogenesis. The invention also describes compounds which bind to and/or activate or inhibit the activity of HB-954 as well as pharmaceutical compositions comprising such compounds. In addition, the invention includes nucleic acid molecules comprising a nucleotide sequence encoding all or a portion of HB-954, gene therapy vectors comprising such sequences, polypeptides comprising all or a portion of HB-954 and antibodies directed against HB-954.

### BACKGROUND OF THE INVENTION

Angiogenesis, the development of new blood vessels from an existing vascular bed, is a complex multistep process that involves the degradation of components of the extracellular matrix and then the migration, proliferation and differentiation of endothelial cells to form tubules and eventually new vessels. Angiogenesis is important in normal physiological processes including, by example and not by way of limitation, embryo implantation; embryogenesis and development; and wound healing. Excessive angiogenesis is also involved in pathological conditions such as tumour cell growth and non-cancerous conditions such as neovascular glaucoma, rheumatoid arthritis, psoriasis and diabetic retinopathy. The vascular endothelium is normally quiescent. However, upon activation, endothelial cells proliferate and migrate to form a primitive tubular network which will ultimately form a capillary bed to supply blood to developing tissues including a growing tumour.

The G-protein-coupled receptors (GPCR) form an important class of peptide-binding receptors. The various members of the GPCR family mediate a wide variety of intercellular signals. Members of the GPCR family have seven helical domains which span the cell membrane and are linked by three extracellular loops and three intracellular loops.

### SUMMARY OF THE INVENTION

The invention provides assays for the identification of compounds useful for the modulation of angiogenesis. Such compounds are useful for the treatment of angiogenesis related diseases. The methods of the invention involve cell-free and cell-based assays that identify compounds which bind to and/or activate or inhibit the activity of HB-954, a G protein-coupled receptor. The assays are optionally followed by an in vivo assay of the effect of the compound on angiogenesis and/or angiogenesis related diseases.

In addition, the invention provides nucleic acid molecules comprising a nucleotide sequence encoding all or a portion of HB-954, polypeptides comprising all or a portion of HB-954, antibodies directed against HB-954.

The invention also describes compounds which bind to and/or activate or inhibit the activity of HB-954 as well as pharmaceutical compositions comprising such compounds.

The invention also provides pharmaceutical compositions comprising a compound identified using the screening methods of the Invention as a well as methods for preparing such compositions by combining such a compound and a pharmaceutically acceptable carrier. Also within the invention are pharmaceutical compositions comprising a compound identified using the screening assays of the invention packaged with instructions for use.

### DETAILED DESCRIPTION OF THE INVENTION

Surprisingly if was found that a GPCR, named HB-954, GenBank Accession number D38449 (see Example 1), has an endothelial preferred pattern of expression, and that levels of its mRNA are induced by two distinct proangiogenic pathways, ie. that of sphingosine-1-phosphate SPP sphingosine-1-phosphate and VEGF (see Table 1).

HB-954 is homologous to the Orexin Receptor family of GPCRs which recogize neuropeptide ligands. Surprisingly, the findings of the present invention now link the endothelial-specific GPCR HB-954, and its putative protein ligand to the biology of endothelial cells, and to the process of angiogenesis.

Hata et al. (Biochimica et Biophysica Acta Vol 1261(1) Mar. 14, 1995 pp 121-125) have originally described the full-length cDNA clone HB-954, isolated from a human fetal brain library. The amino acid sequence of HB-954 deduced by Hata et al. contains four putative glycosylation sites in the N-terminal part, seven presumed transmembrane domains, and a large cytosolic domain in the C-terminal part.

TABLE 1
Relative Levels of endothelial-specific GPCR mRNA expression
detected with the 1834_at probe set on the
Affymetrix HG U95A chip*.

| | relative level of HB-954 mRNA expression | | |
|---|---|---|---|
| tissue sample | (from 3 independent experiments) | | |
| quiescent EC | 89.5 | 35.9 | 10.2 |
| proliferating EC | 92.4 | 123.4 | 29.1 |
| SPP treated EC | 156.1 | 174.4 | 119.9 |
| VEGF treated EC | 171.5 | 140.6 | 126.9 |
| SPP + VEGF | 160.6 | 84.8 | 242.1; |
| treated EC | | | 324.4 |

*Proliferating HUVECs were in continuous culture, all other HUVEC samples were synchronized by overnight incubation in growth factor depleted conditions and then stimulation with one of Sphingosine 1 Phosphate, VEGF or both for 6 hours. Negative values were adjusted to zero. All other cells and tissues tested with the exception of angiopoietin treated smooth muscle cells did not show detectable levels of expression.

Screening Assays

The present invention provides methods for identifying compounds which can be used for the modulation of angiogenesis and for the treatment of a angiogenesis related diseases. The methods entail identifying candidate or test compounds which bind HB-954 and/or have a stimulatory or inhibitory effect on the activity or the expression of HB-954. Preferably, the identification of candidate or test compounds is followed by further determining which of the compounds that bind HB-954 or have a stimulatory or inhibitory effect on the activity or the expression of HB-954 have an effect on angiogenesis in an in vivo assay (effective compounds of the invention).

Candidate or test compounds or agents which bind HB-954 and/or have a stimulatory or inhibitory effect on the activity or the expression of HB-954 are identified in assays that employ either cells which express a form of HB-954 (cell-based assays) or isolated HB-954 (cell-free assays). The various assays of the invention can employ a variety of forms of HB-954, such as full-length HB-954, a biologically active fragment of HB-954, or a fusion protein which includes all or a portion of HB-954.

The assay can be a binding assay entailing direct or indirect measurement of the binding of a test compound or known HB-954 ligand to HB-954.

Thus, in one aspect of the invention there is provided a method for identifying a compound useful for modulating angiogenesis, the method comprising the steps of: a) contacting a test compound with a HB-954 polypeptide and b) determining whether the test compound binds to the HB-954 polypeptide.

Binding of the test compound to the HB-954 polypeptide can be determined either directly or indirectly as described above. In one embodiment, the assay includes contacting the HB-954 polypeptide with a known compound which binds the HB-954 polypeptide to form an assay mixture, contacting the assay mixture with a test compound, and determining the ability of the test compound to interact with the HB-954 polypeptide, wherein determining the ability of the test compound to interact with the HB-954 polypeptide comprises determining the ability of the test compound to preferentially bind to the HB-954 polypeptide as compared to the known compound.

Preferred is a method wherein the binding to the HB-954 polypeptide is within a K_{D} range of 10e⁻⁶ to 10e⁻¹³ preferably within a range of 10e⁻⁸ to 10e⁻¹².

The assay can be in a competitive binding format.

Thus, in a further aspect of the invention there is provided a method for identifying a compound useful for modulating angiogenesis, the method comprising: a) contacting a HB-954 ligand with a HB-954 polypeptide in the presence and absence of a test compound and b) determining whether the test compound alters the binding of the HB-954 ligand to the HB-954 polypeptide.

The assay can also be an activity assay, such as a cellular activity assay, entailing direct or indirect measurement of the activity of HB-954.

Thus, in another aspect of the Invention there is provided a method for identifying a compound useful for modulating angiogenesis, the method comprising: a) contacting a test compound with a cell expressing a HB-954 polypeptide and b) determining whether the test compound alters activity of the HB-954 polypeptide in said cell.

Determining the ability of the test compound to modulate the activity of the membrane-bound form of HB-954 can be accomplished by any method suitable for measuring the activity of HB-954, e.g., any method suitable for measuring the activity of a G-protein coupled receptor or other seven-transmembrane receptor.

The activity of a seven-transmembrane receptor can be measured in a number of ways, not all of which are suitable for any given receptor. Among the measures of activity are: alteration in intracellular Ca²⁺ concentration, activation of phospholipase C, alteration in intracellular inositol triphosphate (IP₃) concentration, alteration in intracellular diacylglycerol (DAG) concentration, and alteration in intracellular adenosine cyclic 3′,5′-monophosphate (cAMP) concentration.

It can also be accomplished, for example, by determining the ability of HB-954 to bind to or interact with a target molecule. The target molecule can be a molecule with which HB-954 binds or interacts with in nature, for example, a molecule on the surface of a cell which expresses HB-954, a molecule on the surface of a second cell, a molecule in the extracellular milieu, a molecule associated with the internal surface of a cell membrane or a cytoplasmic molecule. The target molecule can be a component of a signal transduction a pathway which facilitates transduction of an extracellular signal (e.g., a signal generated by binding of a HB-954 ligand to HB-954) through the cell membrane and into the cell. The target molecule can be, for example, a second intracellular protein which has catalytic activity or a protein which facilitates the association of downstream signaling molecules with HB-954. A HB-954 ligand is one example of a HB-954 target molecule.

The screening assays of the invention may be combined with an in vitro or vivo assay entailing measuring the effect of the test compound on angiogenesis or angiogenesis related diseases.

Thus, the above methods of the invention may further comprise the steps of: c) adding a compound identified by a method of the invention to an assay for modulation of angiogenesis; d) determining whether the compound modulates angiogenesis; and e) identifying a compound that modulates angiogenesis as a compound useful for the treatment of angiogenesis related diseases.

As described in greater detail below, the test compound can be obtained by any suitable means, e.g., from conventional compound libraries. In one embodiment, the invention provides assays for screening candidate or test compounds which bind to or modulate the activity of a membrane-bound form of HB-954. Determining the ability of the test compound to bind to a membrane-bound form of HB-954 can be accomplished, for example, by coupling the test compound with a radioisotope or enzymatic label such that binding of the test compound to the HB-954-expressing cell can be measured by detecting the labeled compound in a complex.

In various embodiments of the above assay methods of the present invention, it may be desirable to immobilize the HB-954 polypeptide (or a HB-954 target molecule) to facilitate separation of complexed from uncomplexed forms of one or both of the proteins, as well as to accommodate automation of the assay. Binding of a test compound to the HB-954 polypeptide, or interaction of the HB-954 polypeptide with a target molecule in the presence and absence of a candidate compound, can be accomplished by methods well known in the art.

The screening assay can also involve monitoring the expression of HB-954. For example, modulators of expression of HB-954 can be identified in a method in which a cell is contacted with a candidate compound and the expression of HB-954 protein or mRNA in the cell is determined. The level of expression of HB-954 protein or mRNA the presence of the cadidate compound is compared to the level of expression of HB-954 protein or mRNA in the absence of the candidate compound. The candidate compound can then be identified as a modulator of expression of HB-954 based on this comparison. For example, when expression of HB-954 protein or mRNA protein is statistically significantly greater in the presence of the candidate compound than in its absence, the candidate compound is identified as a stimulator of HB-954 protein or mRNA expression. Alternatively, when expression of HB-954 protein or mRNA is statistically significantly less in the presence of the candidate compound than in its absence, the candidate compound is identified as an inhibitor of HB-954 protein or mRNA expression. The level of HB-954 protein or mRNA expression in the cells can be determined by methods described below.

Angiogenesis Related Diseases and Angiogenesis Models

“Angiogenesis related diseases” within the meaning of the invention include but are not limited to coronary artery disease, peripheral vascular disease, wound healing, islet cell transplantation, fracture and tendon repair, reconstructive surgery, tissue engineering, restenosis, cancer, age-related macular degeneration, diabetic retinopathy, rheumatoid arthritis, psoriasis, obesity, hemangioma/AIDS-related kaposi's sarcoma, atherosclerotic plaque rupture.

In a preferred embodiment effective compounds identified with the assays of the invention further described herein primarily inhibit the growth of blood vessels and are thus, for example, effective against a number of diseases associated with deregulated angiogenesis, especially diseases caused by ocular neovascularisation, especially retinopathies, such as diabetic retinopathy or age-related macular degeneration, psoriasis, haemangioblastoma, such as haemangioma, mesangial cell proliferative disorders, such as chronic or acute renal diseases, e.g. diabetic nephropathy, malignant nephrosclerosis, thrombotic microangiopathy syndromes or transplant rejection, or especially inflammatory renal disease, such as glomerulonephritis, especially mesangio-proliferative glomerulonephritis, haemolytic-uraemic syndrome, diabetic nephropathy, hypertensive nephrosclerosis, atheroma, arterial restenosis, autoimmune diseases, acute inflammation, fibrotic disorders (e.g. hepatic cirrhosis), diabetes, neurodegenerative disorders and especially neoplastic diseases (solid tumours, but also leukemias and other “liquid-tumours”, especially those expressing c-kit, KDR or flt-1), such as especially breast cancer, cancer of the colon, lung cancer (especially small-cell lung cancer), cancer of the prostate or Kaposi's sarcoma. An effective compound of the invention may inhibit the growth of tumours and is especially suited to preventing the metastatic spread of tumours and the growth of micrometastases.

The angiogenesis modulating activity of the compound can be tested in vitro by a variety of methods such as endothelial cell migration, proliferation, apoptosis, and tube formation. Additionally, more complex ex vivo (Nicosia R. F. and Ottinetti, A. Laboratory Investigation, 63, p115-122, 1990) and in vivo models can be used to assess the activity of angiogenesis modulating compounds (reviewed in Nat Med November 1997;3(11):1203-8). Common models for this include:
1. The matrigel angiogenesis model in which angiogenic: Ancellin N. et al., J. Biol. Chem. 277, 6667-6675, 2002.
2. The corneal pocket assay: Gimbrone, M. A. J., et al., J. Natl. Cancer Inst. 52, 413-427, 1974
3. The chick embryonic chorioallantoic membrane assay: Nguyen, M., et al., Microvascular Research, 47, 31-40, 1994.

The efficacy of the compounds of the invention as it relates to coronary artery disease and peripheral vascular diseases can be modeled as follows. The most commonly used coronary disease models is an ameroid constriction model (Lamping, K A at el., J. Pharmacol Exp. Ther 229, 359-363, 1984). A second model that may mimic the human condition more accurately is a repetitive occlusion model (Kersten J R et al., American J. Physiol. 268, H720-728, 1995). Rabbit, rat, and mouse have been used to model peripheral vascular diseases (Hershey J C et al., Cardiovascular Research 49, 618-625, 2001 and Mack C A et., J. Vascular Surgery, 27, 699-709, 1998).

The efficacy of the compound s of the invention as it relates to age-related macular degeneration or to diabetic retinopathy can be demonstrated in vivo as follows:

In vivo inhibition of choroidal neovascularization is modeled by a laser photocoagulation to rupture Bruch's membrane (Mori et al., American J. of Pathology, 159, 313-320, 2001). Ischemic retinopathy is modeled by first placing neonatal mouse in an hyperoxia environment with subsequent return to normal oxygen tension (Smith L E H et al., Invest. Ophthalmol. Vis. Sci. 35,101-111, 1994).

The antitumor efficacy of the compounds of the invention can be demonstrated in vivo as follows: In vivo activity in the nude mouse xenotransplant model: female BALB/c nude mice (8-12 weeks old), Novartis Animal Farm, Sisseln, Switzerland) are kept under sterile conditions with water and feed ad libitum. Tumors are induced either by subcutaneous injection of tumor cells into mice (for example, Du 145 prostate carcinoma cell line (ATCC No. HTB 81; see Cancer Research 37, 4049-58 (1978)) or by implanting tumor fragments (about 25 mg) subcutaneously into the left flank of mice using a 13-gauge trocar needle under Forene® anaesthesia (Abbott, Switzerland). Treatment with the test compound is started as soon as the tumor has reached a mean volume of 100 mm³. Tumor growth is measured two to three times a week and 24 hours after the last treatment by determining the length of two perpendicular axes. The tumor volumes are calculated in accordance with published methods (see Evans et al., Brit. J. Cancer 45, 466-8 [1982]). The antitumor efficacy is determined as the mean increase in tumor volume of the treated animals divided by the mean increase in tumor volume of the untreated animals (controls) and, after multiplication by 100, is expressed as T/C %. Tumor regression (given in %) is reported as the smallest mean tumor volume in relation to the mean tumor volume at the start of treatment. The test compound is administered daily by gavage.

As an alternative other cell lines may also be used in the same manner, for example:
the MCF-7 breast adenocarcinoma cell line (ATCC No. HTB 22; see also J. Natl. Cancer Inst. (Bethesda) 51, 1409-16 [1973]);
the MDA-MB 468 breast adenocarcinoma cell line (ATCC No. HTB 132; see also In Vitro 14, 911-15 [1978]);
the MDA-MB 231 breast adenocarcinoma cell line (ATCC No. HTB 26; see also J. Natl. Cancer Inst. (Bethesda) 53, 661-74 [1974]);
the Colo 205 colon carcinoma cell line (ATCC No. CCL 222; see also Cancer Res. 38, 1345-55 [1978]);.
the HCT 116 colon carcinoma cell line (ATCC No. CCL 247; see also Cancer Res. 41, 1751-6 [1981]);
the DU145 prostate carcinoma cell line DU 145 (ATCC No. HTB 81; see also Cancer Res. 37, 4049-58 [1978]); and
the PC-3 prostate carcinoma cell line PC-3 (ATCC No. CRL 1435; see also Cancer Res. 40, 524-34 [1980]).

The usefulness of a compound identified by the present invention in the treatment of arthritis as an example of an inflammatory rheumatic or rheumatoid disease can be demonstrated as follows:

The well-known rat adjuvant arthritis model (Pearson, Proc. Soc. Exp. Biol. 91, 95-101 (1956)) is used to test the anti-arthritic activity of compounds of the invention, or salts thereof. Adjuvant Arthritis can be treated using two different dosing schedules: either (i) starting time of immunisation with adjuvant (prophylactic dosing); or from day 15 when the arthritic response is already established (therapeutic dosing). Preferably a therapeutic dosing schedule is used. For comparison, a cyclooxygenase-2 inhibitor, such as 5-bromo-2-(4-fluorophenyl)-3-[4-(methylsulfonyl)phenyl]thiophene or diclofenac, is administered in a separate group.

In detail, male Wistar rats (5 animals per group, weighing approximately 200 g, supplied by Iffa Credo, France) are injected i.d. (intra-dermally) at the base of the tail with 0.1 ml of mineral oil containing 0.6 mg of lyophilised heat-killed Mycobacterium tuberculosis. The rats are treated with the test compound (3, 10 or 30 mg/kg p.o. once per day), or vehicle (water) from day 15 to day 22 (therapeutic dosing schedule). At the end of the experiment, the swelling of the tarsal joints is measured by means of a mico-calliper. Percentage inhibition of paw swelling is calculated by reference to vehicle treated arthritic animals (0% inhibition) and vehicle treated normal animals (100% inhibition).

On the basis of these studies, a compound identified by the present invention is appropriate for the treatment of inflammatory (especially rheumatic or rheumatoid) diseases.

In addition, there exist a number of transgenic models that are useful for angiogenesis and disease-relevant analyses e.g. cancer and cardiovascular diseases (reviewed in Hanahan D. et al., European. J. Cancer 32A,2386-2393, 1996 Carmellet, P. and Collen, D., J. of Pathology, 190, 387-405, 2000).

Test Compounds

Suitable test compounds for use in the screening assays of the invention can be obtained from any suitable source, e.g., conventional compound libraries. The test compounds can also be obtained using any of the numerous approaches in combinatorial library methods known in the art, including: biological libraries; spatially addressable parallel solid phase or solution phase libraries; synthetic library methods requiring deconvolution; the “one-bead one-compound” library method; and synthetic library methods using affinity chromatography selection.

Modeling of Compounds

Computer modeling and searching technologies permit identification of compounds, or the improvement of already identified compounds, that can modulate HB-954 expression or activity. Having identified such a compound or composition, the active sites or regions are identified. Such active sites might typically be ligand binding sites. The active site can be identified using methods known in the art including, for example, from the amino acid sequences of peptides, from the nucleotide sequences of nucleic acids, or from study of complexes of the relevant compound or composition with its natural ligand. In the latter case, chemical or X-ray crystallographic methods can be used to find the active site by finding where on the factor the complexed ligand is found.

Next, the three dimensional geometric structure of the active site is determined. This can be done by known methods, including X-ray crystallography, which can determine a complete molecular structure. On the other hand, solid or liquid phase NMR can be used to determine certain intramolecular distances. Any other experimental method of structure determination can be used to obtain partial or complete geometric structures. The geometric structures may be measured with a complexed ligand, natural or artificial, which may increase the accuracy of the active site structure determined.

Finally, having determined the structure of the active site, either experimentally, by modeling, or by a combination, candidate modulating compounds can be identified by searching databases containing compounds along with information on their molecular structure. Such a search seeks compounds having structures that match the determined

1. A method for identifying a compound useful for modulating angiogenesis, the method comprising the steps of: a) contacting a test compound with a HB-954 polypeptide; and b) determining whether the test compound binds to the HB-954 polypeptide.

2. The method of claim 1, wherein the binding to the HB-954 polypeptide is within a K_{D} range of 10e⁻⁶ to 10e⁻¹³ preferably within a range of 10e⁻⁸ to 10e^{-12.}

3. The method of claim 1, said method further comprising the steps of: c) adding a compound identified as binding to the HB-954 polypeptide in step (b) to an assay for the modulation of angiogenesis; d) determining whether the compound modulates angiogenesis; and e) identifying a compound that modulates angiogenesis in step (d) as a compound useful for the treatment of angiogenesis related diseases.

4. A method for identifying a compound useful for modulating angiogenesis, the method comprising: a) contacting a HB-954 ligand with a HB-954 polypeptide in the presence and absence of a test compound; and b) determining whether the test compound alters the binding of the HB-954 ligand to the HB-954 polypeptide.

5. The method of claim 4, said method further comprising the steps of: c) adding a compound identified that alters binding of the HB-954 ligand to the HB-954 polypeptide in step (b) to an assay for the modulation of angiogenesis; d) determining whether the compound modulates angiogenesis; and e) identifying a compound that modulates angiogenesis in step (d) as a compound useful for the treatment of angiogenesis related diseases.

6. The method of claims 1, wherein the HB-954 polypeptide is expressed on the surface of a recombinant cell.

7. The method of claim 6, wherein said recombinant cell is an eukaryotic cell.

8. A method for identifying a compound useful for modulating angiogenesis, the method comprising: a) contacting a test compound with a cell expressing a HB 954 polypeptide; and b) determining whether the test compound alters activity of the HB-954 polypeptide in said cell;

9. The method of claim 8, said method further comprising the steps of: c) adding a compound identified that alters the activity of the HB-954 polypeptide in step (b) to an assay for the modulation of angiogenesis; d) determining whether the I compound modulates angiogenesis; and e) identifying a compound that modulates angiogenesis in step (d) as a compound useful for the treatment of angiogenesis related diseases.

10. The method of claim 1, wherein modulation of angiogenesis is inhibition of angiogenesis.

11. The method of claim 1, wherein modulation of angiogenesis is activation of angiogenesis.

12. The method of claim 8 wherein the activity of the HB-954 polypeptide is determined by measuring the level of cAMP in the cell.

13. The method of claim 8, wherein the activity of the HB-954 polypeptide is determined by measuring the level of cytoplasmic Ca²⁺ in the cell.

14. The method of claim 8, wherein which the cell further contains a reporter gene operatively associated with a cAMP responsive element, and the level of cAMP is measured by measuring expression of the reporter gene.

15. The method of claim 8, wherein the activity of the HB-954 polypeptide is measured by measuring intracellular inosital 1,4,5-trisphophate (IP₃).

16. The method of claim 8, wherein the activity of HB-954 is measured by measuring intracellular 1,2-diacyiglycerol (DAG).

17. A pharmaceutical formulation for the modulation of angiogenesis, comprising a compound that modulates the activity of HB-954, mixed with a pharmaceutically acceptable carrier.

18. A package comprising the pharmaceutical formulation of claim 17 and instructions for administering the pharmaceutical formulation for the purpose of modulating angiogenesis.

19. A gene therapy vector comprising a nucleic acid molecule that encodes HB-954 protein or a biologically active fragment thereof.

20. A nucleic acid molecule that is complementary to a nucleic acid molecule that encodes HB-954 protein or a fragment thereof.

21. A gene therapy vector comprising the nucleic acid molecule of claim 20.

22. The use of HB-954 protein or a biologically active fragment thereof in medicine.

23. The use of a monoclonal antibody which specifically binds an epitope of HB-954 protein or a biologically active fragment thereof in medicine.
